(19) United States
(12) Patent Application Publication
(10) Pub. No.: US 2015/0367107 A1
(43) Pub. Date: Dec. 24, 2015

(54) Balloon Catheter With Matte Distal Tip

(71) Applicant: Cook Medical Technologies LLC, Bloomington, IN (US)

(72) Inventor: Stephanie RIVERA, Burlington, NC (US)

(21) Appl. No.: 14/309,279

(22) Filed: Jun. 19, 2014

(51) Int. Cl. A61M 25/10

(52) U.S. Cl. CPC A61M 25/1002; A61M 25/1025; A61M 25/1034; A61M 2025/1088

(57) ABSTRACT

A balloon catheter using a non-latex balloon is disclosed. The balloon catheter includes a catheter having a polished outer surface and a balloon section having a matte outer surface. A lumen extends from a proximal end of the catheter to the balloon section and an inflation port provides fluid communication between the lumen and the outer surface of the balloon section. A balloon is positioned over the balloon section and secured at a distal and proximal end.

### FIELD

Embodiments of the present invention relate to medical devices and more particularly to devices and methods for inflating a balloon having a tacky surface at a distal end of a catheter.

### BACKGROUND

A variety of tracts or ducts in the body are subject to the development of stones, calculi or the like. (For convenience, such stones, calculi and the like may sometimes be referred to herein by the words “stone” or “stones”). For example, stones may develop in the kidneys and migrate down the ureters. Sometimes such stones become lodged in the ureters, requiring intervention for their removal. Similarly, gallstones may develop in the gallbladder, and migrate down the biliary duct (the common bile duct), through the ampulla of Vater, and out the Sphincter of Oddi into the duodenum. As with kidney stones, such stones occasionally become lodged in the biliary duct, the pancreatic duct or the ampulla of Vater. Indeed, gallstones can be of such a size as to be unable to pass through the Sphincter of Oddi. In either case, intervention is again required for their removal.

Currently, stones may be removed through the use of an extraction balloon. The extraction balloon is delivered past the location of the stone, inflated, and then retrieved through the duct, removing the stone in the process. Currently available extraction balloons are typically made of latex. However, some patients are allergic to latex and consequently, cannot be treated using a latex balloon.

Silicone has been identified as a suitable replacement for latex in many applications and potentially could replace latex as an extraction balloon. However, the use of silicone has its own potential shortcomings. For one, silicone is generally tacky and tends to stick to other materials, including materials that it is packaged with. For example, in a balloon catheter having a silicone balloon, the silicone balloon will tend to adhere to an underlying catheter. This is potentially a problem, as the balloon may inflate unevenly during a procedure. In some instances, the silicone balloon may inflate only in the region adjacent an inflation port, while remaining adhered to the catheter at other locations.

FIG. 1 illustrates a partially inflated balloon catheter 100 in which an inner surface of a balloon 102 is adhered to an outer surface of a catheter 104. The balloon 102 has inflated in an area immediately adjacent an inflation port 106, but is otherwise adhered to the catheter 104 in the other areas. As the balloon 102 is inflated further the inflation pressure may overcome the adhesion between the outer surface of the catheter 104 and the inner surface of the balloon 102, or the balloon 102 may continue to inflate only adjacent the inflation port 106 eventually failing. Both outcomes are unsatisfactory and the use of a silicone balloon, or any balloon that adheres to the outer surface of the catheter such as thermoplastic elastomers, is therefore limited in application.

It would be beneficial to develop an alternative extraction balloon that does not use latex, but that inflates evenly without adhering to the tip of a balloon catheter.

### SUMMARY

Embodiments of the invention include an extraction balloon catheter. The extraction balloon catheter is comprised of a tubular member and a balloon. The tubular member has a longitudinal lumen, an outer surface, a proximal section, a balloon section, and a distal section. The outer surface of the proximal section has a polished finish and the outer surface of the balloon section has a matte finish. A port is disposed in the balloon section, the port providing fluid communication between the longitudinal lumen and the outer surface of the of the balloon section. The balloon is disposed about the balloon section of the tubular member and is secured to the outer surface at a proximal balloon end and to the outer surface at a distal balloon end.

Another embodiment includes a method for manufacturing a balloon extraction catheter. In the method a catheter having a lumen is divided into a balloon section and a non-balloon section. The surface finish of the balloon section of the catheter is modified to have a matte finish. A port is formed in the catheter with the port providing fluid communication between the outer surface of the balloon section and the longitudinal lumen. A balloon is placed over the balloon section of the catheter and a distal end of the balloon is secured to the catheter. A proximal end of the balloon is then secured to the catheter.

In another embodiment, an extraction balloon catheter comprises a tubular member and a balloon. The tubular member has a longitudinal lumen and a balloon section. The tubular member has a first outer surface with a first average surface irregularity and the balloon section has a second outer surface with a second average surface irregularity. The first average surface irregularity is less than the second average surface irregularity. A port is disposed in the balloon section with the port providing fluid communication between the longitudinal lumen and the second outer surface. The balloon is disposed about the balloon section of the tubular member and is secured to the catheter at a proximal balloon end and to the catheter at a distal balloon end.

### BRIEF DESCRIPTION OF THE DRAWINGS

To further clarify the above and other advantages and features of the one or more present inventions, reference to specific embodiments thereof are illustrated in the appended drawings. The drawings depict only typical embodiments and are therefore not to be considered limiting. One or more embodiments will be described and explained with additional specificity and detail through the use of the accompanying drawings in which:

FIG. 1 is a side view of a distal end of a partially inflated prior art balloon catheter showing the outer surface of the catheter adhering to the balloon.

FIG. 2 is a side view of a distal end of an embodiment of an extraction balloon catheter according to the present invention.

FIG. 3 is a detailed view of the surface of the catheter of FIG. 2.

FIG. 4 is a side view of the distal end of the embodiment of the extraction balloon catheter of FIG. 2 with the balloon inflated.

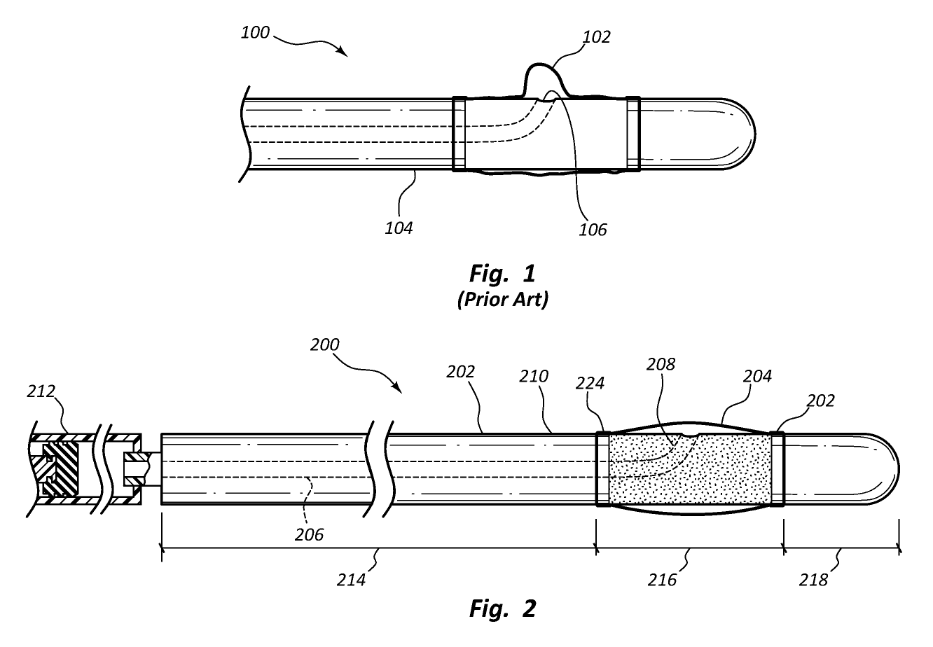

The drawings are not necessarily to scale.

### DETAILED DESCRIPTION

As used herein, “at least one,” “one or more,” and “and/or” are open-ended expressions that are both conjunctive and disjunctive in operation. For example, each of the expressions “at least one of A, B and C,” “at least one of A, B, or C,” “one or more of A, B, and C,” “one or more of A, B, or C” and “A, B, and/or C” means A alone, B alone, C alone, A and B together, A and C together, B and C together, or A, B and C together.

Various embodiments of the present inventions are set forth in the attached figures and in the Detailed Description as provided herein and as embodied by the claims. It should be understood, however, that this Detailed Description does not contain all of the aspects and embodiments of the one or more present inventions, is not meant to be limiting or restrictive in any manner, and that the invention(s) as disclosed herein is/are and will be understood by those of ordinary skill in the art to encompass obvious improvements and modifications thereto.

Additional advantages of the present invention will become readily apparent from the following discussion, particularly when taken together with the accompanying drawings.

In the following discussion, the terms “distal” and “proximal” will be used to describe the opposing axial ends of the inventive balloon catheter, as well as the axial ends of various component features. The term “distal” is used in its conventional sense to refer to the end of the apparatus (or component thereof) that is furthest from the operator during use of the apparatus. The term “proximal” is used in its conventional sense to refer to the end of the apparatus (or component thereof) that is closest to the operator during use. For example, a catheter may have a distal end and a proximal end, with the proximal end designating the end closest to the operator heart during an operation, such as a handle, and the distal end designating an opposite end of the catheter, such as treatment tip. Similarly, the term “distally” refers to a direction that is generally away from the operator along the apparatus during use and the term “proximally” refers to a direction that is generally toward the operator along the apparatus.

FIG. 2 illustrates a side view of an embodiment of an extraction balloon catheter 200 according to the present invention. The extraction balloon catheter 200 is comprised of a tubular member, such as a catheter 202, and a balloon 204. The balloon 204 is formed of an elastic material that tends to adhere to the catheter 202. Examples of such materials include silicone and thermoplastic elastomers. The balloon 204 is generally disposed at a distal end of the catheter 202 and is secured to the catheter 202 at a proximal end 224 of the balloon 204 and a distal end 222 of the balloon 204.

The catheter 202 has a lumen 206 extending longitudinally from an area proximate the proximal end of the catheter 202 to an area proximate the distal end of the catheter 202. An inflation port 208 provides a fluid communication path between the lumen 206 and an outer surface 210 of the catheter 202. Although not shown, it is possible to have multiple lumens in the catheter 202 for guiding the catheter 202 or deploying other medical devices. A syringe 212 is shown in fluid commination with the lumen 206, such that the syringe 212 may be used as an inflation device. Other inflation devices are possible and embodiments of the invention are not limited to the use of a syringe 212.

The catheter 202 may be formed of multiple sections of material or may be a single material as shown in FIG. 2. The catheter 220 of FIG. 2 has three distinct sections comprising a proximal section 214, a balloon section 216, and a distal section 218. The proximal section 214 and the distal section 218 may have a polished finish with low surface irregularities. The balloon section 216 contains the inflation port and has a matte finish having higher surface irregularities. The polished finish may be necessary to reduce trauma to a patient and inhibit microorganisms from colonizing on the catheter 202.

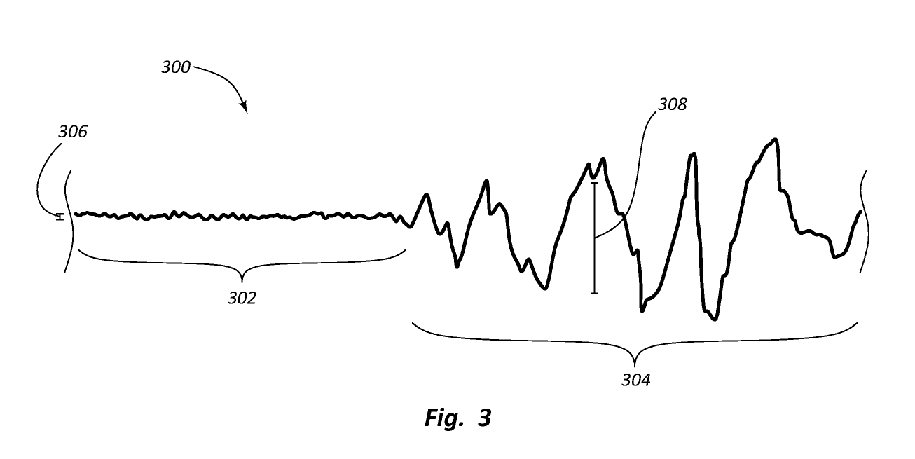

FIG. 3 is a close up view of a surface 300 of a catheter illustrating the difference between a polished finish 302 and a matte finish 304. In the area having a polished finish 302, there are relatively little surface irregularities. One method of measuring the surface irregularities is to measure a depth of the surface irregularities and find the average surface deviation. As shown in FIG. 3, the average surface deviation 306 of the polished section 302 is less than the average surface deviation 308 of the matte section 304. Other methods of quantifying the surface irregularities or finish are well known and the average surface deviation is only given to be an example.

In one embodiment the average surface deviation of the polished surface may be 3 nanometers while the matte surface may have an average surface deviation of 12 nanometers. In other embodiments the average surface deviation of the matte finish may be at least triple the average surface deviations of the polished section.

The balloon 204 is positioned about the catheter 202 such that the balloon 204 is disposed over the balloon section 216. The balloon 204 is secured to the catheter 202 at the balloon's distal 222 and proximal ends 224, leaving a middle portion disposed over the inflation port 208. In some embodiments the balloon 204 may be secured to the catheter 202 within the balloon section 216, or in other embodiments the balloon 204 may be secured external to the balloon section 216, or a combination of the two.

When an inflation fluid is delivered to the interior volume of the balloon 204 through the inflation port 208, the balloon 204 begins to inflate. The matte finish of the balloon portion 216 inhibits the balloon 204 from adhering to the catheter 202, such that the balloon 204 inflates more readily than if it were adhered to the catheter surface. The irregular surface also may allow fluid to flow between the irregularities further enhancing inflation of the balloon 204 when the balloon 204 is adjacent the catheter 202.

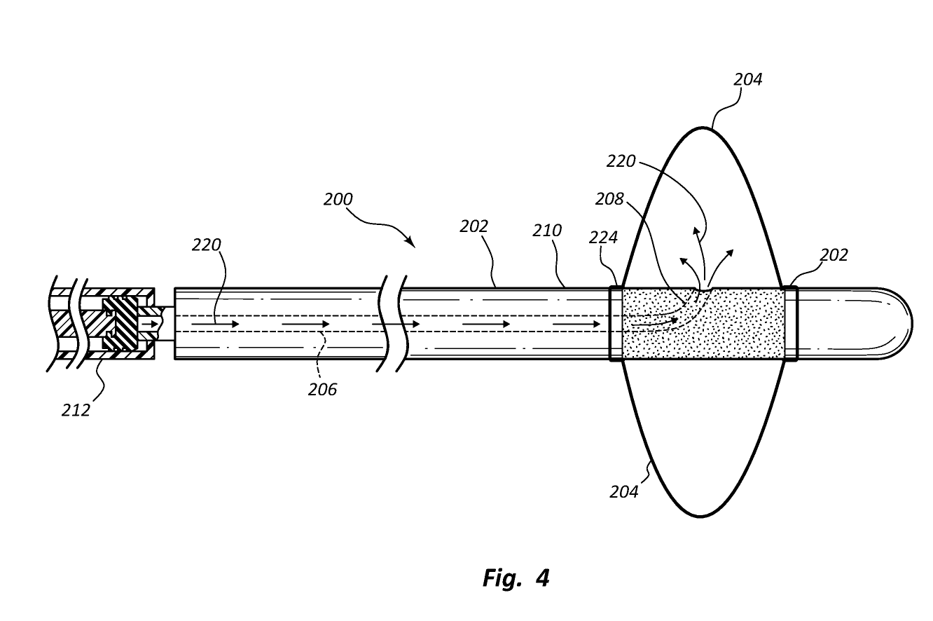

FIG. 4 illustrates the extraction balloon catheter 200 of FIG. 2 with the balloon 204 in an inflated state. Inflation fluid, illustrated by arrows 220 delivered through the inflation port 208 inflates the balloon 204. The inflation fluid may be metered by the inflation fluid source to inject a known amount of fluid to inflate the balloon 204 to a specific size. For instance, if a syringe is the inflation source, the plunger may be advanced to a mark to deliver a specific amount of inflation fluid.

The process of manufacturing extraction balloon catheter 200 will now be described with references to FIG. 2. Initially, a catheter 202 having a longitudinal lumen 206 is divided into a balloon section 216 and a non-balloon section 214, 218. The surface of the balloon section 216 is then modified to have a matte finish. An inflation port 208 is formed in the catheter 202 to provide fluid communication between the outer surface of the balloon section 216 and the longitudinal lumen 206. A balloon 204 is then placed over the matte finish of the balloon section 216. A distal end 222 of the balloon 204 is secured to the catheter 202 and a proximal end 224 is secured to the catheter 202.

The balloon section 216 may be modified to have a matte finish through a variety of processes. In one embodiment the surface may be modified by buffing the surface. In another embodiment the balloon section 216 may be blasted by particles to form the matte finish. For example, a stream of plastic pellets could be used to form the matte finish. In another embodiment the matte finish is formed by etching the surface of the balloon section 216. An etching solution may be applied to the balloon section 216 to form the matte finish.

In some embodiments the outer surface of the catheter 202 may be polished by passing the catheter 202 through a heated die having a polished surface. The catheter 202 may be heated above its melting temperature and passed through the heated die, decreasing the size of the catheter 202 and polishing the outer surface. This procedure may be done prior to modifying the surface of the balloon section 216.

Embodiments of the invention have been primarily described in relation to a catheter and a balloon. It should be understood that various changes and modifications to the presently preferred embodiments described herein will be apparent to those skilled in the art. Such changes and modifications can be made without departing from the spirit and scope of the present invention and without diminishing its intended advantages. It is therefore intended that such changes and modifications be covered by the appended claims.

What is claimed:

1. A balloon catheter comprising:
a tubular member having a longitudinal lumen disposed therein, the tubular member having an outer surface, a proximal section, a balloon section, and a distal section, the outer surface of the proximal section having a polished finish and the outer surface of the balloon section having a matte finish;
a port disposed in the balloon section, the port providing fluid communication between the longitudinal lumen and the outer surface of the of the balloon section; and
a balloon disposed about the balloon section of the tubular member, the balloon being secured to the outer surface at a proximal balloon end and to the outer surface at a distal balloon end.

2. The balloon catheter of claim 1 wherein the outer surface of the distal section has a polished finish.

3. The balloon catheter of claim 1 wherein the polished finish is defined as a surface having less surface irregularities than the matte finish.

4. The balloon catheter of claim 1 wherein the polished finish is defined as a surface having relatively low surface irregularities.

5. The balloon catheter of claim 1 wherein the matte finish is defined as a surface having relatively high surface irregularities.

6. The balloon catheter of claim 2 wherein the surface irregularities of the matte finish are at least triple the surface irregularities of the polished finish as measured by the average surface deviation.

7. The balloon catheter of claim 1 further comprising an inflation source fluidly coupled to the longitudinal lumen.

8. The balloon catheter of claim 1 further comprising a second longitudinal lumen adapted to receive a guidewire.

9. A method of manufacturing a balloon catheter, the method comprising:
dividing a catheter having a longitudinal lumen into a balloon section and a non-balloon section;
modifying the surface finish of the balloon section of the catheter to have a matte finish;
forming a port in the catheter, the port providing fluid communication between the outer surface of the balloon section and the longitudinal lumen;
placing a balloon over the balloon section of the catheter;
securing a distal end of the silicone balloon to the catheter; and
securing a proximal end of the silicone balloon to the catheter.

10. The method of claim 9 wherein the balloon is comprised of silicone.

11. The method of claim 9 wherein the balloon section is on a distal end of the catheter.

12. The method of claim 9 wherein the surface finish of the balloon section is modified through a process selected from the group consisting of buffing, blasting, and etching.

13. The method of claim 9 further comprising polishing an outer surface of the catheter by passing the catheter through a heated die.

14. The method of claim 13 wherein the outer surface of the catheter is polished prior to passing the catheter through the heated die.

15. The method of claim 9 wherein the distal end of the balloon is secured to the catheter at a distal end of the balloon section and the proximal end of the balloon is secured to the catheter at a proximal end of the balloon section.

16. The method of claim 9 wherein the balloon section is proximal a distal end of the catheter and the catheter has a polished outer surface distal to the balloon section.

17. A balloon catheter comprising:
a tubular member having a longitudinal lumen disposed therein, the tubular member having a balloon section, the tubular member having a first outer surface with a first average surface irregularity and the balloon section having a second outer surface with a second average surface irregularity, wherein the first average surface irregularity is less than the second average surface irregularity;
a port disposed in the balloon section, the port providing fluid communication between the longitudinal lumen and the second outer surface; and
a balloon disposed about the balloon section of the tubular member, the balloon being secured to the catheter at a proximal balloon end and to the catheter at a distal balloon end.

18. The balloon catheter of claim 17 wherein the proximal balloon end and the distal balloon end are secured to the catheter outside of the balloon section.

19. The balloon catheter of claim 17 wherein the proximal balloon end and the distal balloon end are secured to the catheter within the balloon section.

20. The balloon catheter of claim 17 wherein the first outer surface has a polished finish and the second outer surface has a matte finish.
